(19) United States
(12) Patent Application Publication
(10) Pub. No.: US 2023/0406869 A1
(43) Pub. Date: Dec. 21, 2023

(54) COMPOUND, METHOD FOR PRODUCING COMPOUND, AND METHOD FOR PRODUCING SURFACE TREATMENT AGENT

(71) Applicant: AGC Inc., Tokyo (JP)

(72) Inventors: Makoto UNO, Chiyoda-ku (JP); Eiichiro ANRAKU, Chiyoda-ku (JP); Takafumi KAWAKAMI, Chiyoda-ku (JP); Keigo MATSUURA, Chiyoda-ku (JP)

(73) Assignee: AGC Inc., Tokyo (JP)

(21) Appl. No.: 18/460,114

(22) Filed: Sep. 1, 2023

Related U.S. Application Data
(63) Continuation of application No. PCT/JP2022/008824, filed on Mar. 2, 2022.

(30) Foreign Application Priority Data
Mar. 5, 2021 (JP) 2021-035095

(51) Int. Cl. C07F 7/18

(52) U.S. Cl. CPC C07F 7/1876

(57) ABSTRACT

A compound represented by the following formula (A1) or (A2):

G¹-(CH₂)ₘ₁—CHX¹—(CH₂)ₙ₁-M Formula (A1)

M-(CH₂)ₙ₂—CHX¹—(CH₂)ₘ₂-G²(CH₂)ₘ₃—CHX¹—(CH₂)ₙ₃-M Formula (A2).

Where in the formula (A1) and (A2), G¹, G², X¹, M, n1, n2, n3, m1, m2, and m3 are as defined in the disclosure. A method for producing a compound represented by the following formula (B1) or (B2), where M in the compound of formula (A1) or (A2) is converted into a hydrogen or hydrocarbon group represented by R³:

G¹-(CH₂)ₘ₁—CHX¹—(CH₂)ₙ₁—R³ Formula (B1)

R³—(CH₂)ₙ₂—CHX¹—(CH₂)ₘ₂-G²-(CH₂)ₘ₃—CHX¹—(CH₂)ₙ₃—R³ Formula (B2).

Where in the formulas B1 and B2, G¹, G², X¹, R³, n1, n2, n3, m1, m2, and m3 are as defined in the disclosure.

### INCORPORATION BY REFERENCE

This application is based upon and claims the benefit of priority from Japanese Patent Application 2021-35095 filed on Mar. 5, 2021, and PCT application No. PCT/JP2022/008824 filed on Mar. 2, 2022, the disclosure of which is incorporated herein in its entirety by reference.

### BACKGROUND

The present invention relates to a compound, a method for producing a compound, and a method for producing a surface treatment agent.

Fluorine-containing compounds are used in various fields such as agricultural chemicals, pharmaceuticals, and functional materials. It is required to synthesize various structures by a simpler method. For example, in order to impart various functions to a compound having a fluoroalkyl group or a (poly)oxyfluoroalkylene chain, synthesis methods for introducing various substituents have been studied.

Various studies have been made on a method for synthesizing a compound having a structure in which an alkyl group is bonded to a fluoroalkyl group.

For example, Japanese Unexamined Patent Application Publication No. 2018-43940 discloses a method for producing a fluorine-containing compound by adding a perfluoroalkyl bromide to an olefin compound by a radical reaction.

### SUMMARY

The technique of Japanese Unexamined Patent Application Publication No. 2018-43940 is not suitable for synthesis of a compound having a carbon-carbon double bond because an olefin is reacted, and the type of electrophile is limited. In addition, since the product can further undergo a radical reaction to be telomerized, there is a problem that various types of by-products are generated.

An object of the present invention is to provide a novel compound, a method for producing the same, a method for producing another compound using the novel compound as a raw material, and a method for producing a surface treatment agent.

The present invention provides a compound, a method for producing the same, and a method for producing a surface treatment agent having the following configurations [1] to [7].
[1] A compound represented by the following formula (A1) or (A2).

G¹-(CH₂)ₘ₁—CHX¹—(CH₂)ₙ₁-M Formula (A1)

M-(CH₂)ₙ₂—CHX¹—(CH₂)ₘ₂-G²-(CH₂)ₘ₃—CHX¹—(CH₂)ₙ₃-M Formula (A2)
where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
X¹ is a halogen atom, and when there are a plurality of X¹'s, the X¹'s may be the same as or different from each other,
M is BR¹R², R¹ and R² are each independently a group represented by OH, —R¹¹, —OR¹¹, —O—C(O)—R¹¹, or —NR¹¹R¹², R¹¹ is a hydrocarbon group which may have a substituent or a heteroatom in a carbon chain, two R¹¹'s may be linked to form a ring structure, R¹² is a hydrogen atom or an alkyl group having 1 to 6 carbon atoms, and when there are a plurality of M's, the M's may be the same as or different from each other,
n1, n2, and n3 are each independently an integer of 0 to 20, and
m1, m2, and m3 are each independently 1 or 2.
[2] A method for producing a compound represented by the following formula (B1) or (B2), in which M in the compound of [1] is converted into hydrogen or a hydrocarbon group.

G¹-(CH₂)ₘ₁—CHX¹—(CH₂)ₙ₁—R³ Formula (B1)

R³—(CH₂)ₙ₂—CHX¹—(CH₂)ₘ₂-G²-(CH₂)ₘ₃—CHX¹—(CH₂)ₙ₃—R³ Formula (B2)
where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
X¹ is a halogen atom, and when there are a plurality of X¹'s, the X¹'s may be the same as or different from each other,
R³ is a hydrogen atom or a hydrocarbon group which may have a substituent or a heteroatom, and when there are a plurality of R³'s, the R³'s may be the same as or different from each other, n1, n2, and n3 are each independently an integer of 0 to 20, and
m1, m2, and m3 are each independently 1 or 2.
[3] The method for producing a compound represented by the formula (B1) or (B2), which is the production method according to [2], the method comprising: reacting the compound according to [1] with the compound represented by the following formula (C1).

R³—X² Formula (C1)
where, in the formula,
R³ is a hydrocarbon group which may have a substituent or a heteroatom, and X² is a halogen atom.
[4] A method for producing a compound represented by the following formula (B3) or (B4), in which X¹ of the compound according to [1] is converted into a hydrogen or hydrocarbon group.

G¹-(CH₂)ₘ₁—CHR³—(CH₂)ₙ₁-M Formula (B3)

M-(CH₂)ₙ₂—CHR³—(CH₂)ₘ₂-G²-(CH₂)ₘ₃—CHR³—(CH₂)ₙ₃-M Formula (B4)
where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
M is BR¹R², R¹ and R² are each independently a group represented by —OH, —R¹¹, —OR¹¹, —O—C(O)—R¹¹, or —NR¹¹R¹², R¹¹ is a hydrocarbon group which may have a substituent or a heteroatom in a carbon chain, two R¹¹'s may be linked to form a ring structure, R¹² is a hydrogen atom or an alkyl group having 1 to 6 carbon atoms, and when there are a plurality of M's, the M's may be the same as or different from each other,
R³ is a hydrogen atom or a hydrocarbon group which may have a substituent or a heteroatom, and when there are a plurality of R³'s, the R³'s may be the same as or different from each other, and n1, n2, and n3 are each independently an integer of 0 to 20, and
m1, m2, and m3 are each independently 1 or 2.
[5] The method for producing a compound represented by the formula (B3) or (B4), which is the production method according to [4], the method including: reacting the compound according to [1] with the compound represented by the following formula (C2).

R³—MgR⁴ Formula (C2)
where, in the formula,
R³ is a hydrocarbon group which may have a substituent or a heteroatom, and R⁴ is a halogen atom or a hydrocarbon group which may have a substituent or a heteroatom.
[6] A method for producing the compound according to [1], the method including: reacting a compound represented by the following formula (E1) or (E2) with a compound represented by the following formula (H1).

G¹-(CH₂)ₘ₄—X¹ Formula (E1)

X¹—(CH₂)ₘ₅-G²-(CH₂)ₘ₆—X¹ Formula (E2)

CH₂═CH—(CH₂)ₙ-M Formula (H1)
where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
X¹ is a halogen atom, and when there are a plurality of X¹'s, the X¹'s may be the same as or different from each other,
M is BR¹ R², R¹ and R² are each independently a group represented by —OH, —R¹¹, —OR¹¹, —O—C(O)—R¹¹, or —NR¹¹R¹², R¹¹ is a hydrocarbon group which may have a substituent or a heteroatom in a carbon chain, two R¹¹'s may be linked to form a ring structure, R¹² is a hydrogen atom or an alkyl group having 1 to 6 carbon atoms, and when there are a plurality of M's, the M's may be the same as or different from each other,
n is an integer of 0 to 20, and
m4, m5, and m6 are each independently 0 or 1.
[7] A method for producing a surface treatment agent, the method comprising: obtaining a compound represented by the formula (B1), (B2), (B3), or (B4) by the production method according to any one of [2] to [5]; and introducing a reactive silyl group into the compound.

The above and other objects, features and advantages of the present disclosure will become more fully understood from the detailed description given hereinbelow which are given by way of illustration only, and thus are not to be considered as limiting the present disclosure.

The present invention provides a novel compound, a method for producing the same, a method for producing another compound using the novel compound as a raw material, and a method for producing a surface treatment agent.

### DESCRIPTION OF EMBODIMENTS

In the present specification, the compound represented by the formula (A1) is referred to as a compound (A1). The same applies to compounds represented by other formulae and the like.

The “(poly)oxyfluoroalkylene” is a generic term for oxyfluoroalkylene and polyoxyfluoroalkylene.

The fluoroalkyl group is a generic term for a combination of a perfluoroalkyl group and a partial fluoroalkyl group. The perfluoroalkyl group means a group in which all hydrogen atoms of the alkyl group are substituted with fluorine atoms. In addition, the partial fluoroalkyl group is an alkyl group in which one or more hydrogen atoms are substituted with a fluorine atom and which has one or more hydrogen atoms.

That is, the fluoroalkyl group is an alkyl group having one or more fluorine atoms.

The “reactive silyl group” is a generic term for a hydrolyzable silyl group and a silanol group (Si—OH), and the “hydrolyzable silyl group” means a group capable of forming a silanol group by a hydrolysis reaction.

The “surface layer” means a layer formed on the surface of the substrate.

“to” indicating a numerical range means that the numerical values stated before and after “to” are included as a lower limit value and an upper limit value.

### Compound (A1) and compound (A2)

The compound according to the present invention (hereinafter also referred to as the present compound) is a compound represented by the following formula (A1) or (A2).

G¹-(CH₂)ₘ₁—CHX¹—(CH₂)ₙ₁-M Formula (A1)

M-(CH₂)ₙ₂—CHX¹—(CH₂)ₘ₂-G²-(CH₂)ₘ₃—CHX¹—(CH₂)ₙ₃-M Formula (A2)
where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
X¹ is a halogen atom, and when there are a plurality of X¹'s, the X¹'s may be the same as or different from each other,
M is BR¹R², R¹ and R² are each independently a group represented by —OH, —R¹¹, —OR¹¹, —O—C(O)—R¹¹, or —NH—R¹¹, and may be hydrocarbon group which may have a substituent or a heteroatom in a carbon chain, two R¹¹'s may be linked to form a ring structure, and when there are a plurality of M's, the M's may be the same as or different from each other,
n is an integer of 0 to 20, and
m1, m2, and m3 are each independently 1 or 2.

The present compound has a structure in which a fluoroalkyl chain or a (poly)oxyfluoroalkylene chain and an alkyl chain are linked by a carbon-carbon bond or an oxygen-carbon bond without any bond such as an ester bond and an amide bond, and the alkyl chain has a halogen atom (X¹) and a boron-based substituent (M) as substituents.

As described above, the present compound is excellent in chemical stability because a fluoroalkyl chain or a (poly)oxyfluoroalkylene chain and an alkyl chain are linked without any bond. In addition, in the present compound, the alkyl chain has two types of substituents (X¹ and M) with different reactivities. The present compound having such a structure is a preferred compound as a raw material for obtaining a fluoroalkyl chain or a (poly)oxyfluoroalkylene chain to which a substituent is easily introduced at the X¹ or M position and various functionalities are imparted by a one-step or multi-step reaction. The present compound can be used as an intermediate for pharmaceuticals, agricultural chemicals, resins, coating agents, and the like, in addition to a surface treatment agent.

The fluoroalkyl group in G¹ may be a linear fluoroalkyl group or a fluoroalkyl group having a branched or ring structure. The number of carbon atoms in the fluoroalkyl group may be appropriately adjusted according to the application of the present compound. From the viewpoint of ease of production of the present compound and an increase in yield in the production of other compounds using the present compound as a raw material, the number of carbon atoms is preferably 1 to 30, more preferably 1 to 20, still more preferably 1 to 10, and particularly preferably 1 to 6.

Specific examples of the fluoroalkyl group include CF₃—, CHF₂—, CF₃CF₂—, CF₃CHF—, CF₃CF₂CF₂—, CF₃CHFCF₂—, CF₃CHFCHF—, CF₃CF(CF₃)—, CF₃CF₂CF₂CF₂—, CF₃CHFCF₂CF₂—, CF₃CF(CF₃)—CF₂—, CF₃C(CF₃)₂—CF₂—, CF₃CF₂CF₂CF₂CF₂—, CF₃ CF₂CF₂CF₂CF₂CF₂—, a fluorocyclobutyl group, a fluorocyclopentyl group, and a fluorocyclohexyl group.

The monovalent group having a (poly)oxyfluoroalkylene chain in G¹ is a fluoroalkyl group having —O— at a terminal bonded to CH₂, —O— between carbon-carbon atoms of a carbon chain having 2 or more carbon atoms, or both of them in the formula (A1). From the viewpoint of ease of production and the like, G¹ preferably has a structure represented by the following formula (G1-1).

R^{f0}O—[(R^{f1}O)ₘ₁₁(R^{f2}O)ₘ₁₂(R^{f3}O)ₘ₁₃(R^{f4}O)ₘ₁₄(R^{f5}O)ₘ₁₅(R^{f6}O)ₘ₁₆]—(R^{f7})ₘ₁₇— Formula (G1-1)
where
R^{f0} is a fluoroalkyl group having 1 to 20 carbon atoms,
R^{f1} is a fluoroalkylene group having 1 carbon atom,
R^{f2} is a fluoroalkylene group having 2 carbon atoms,
R^{f3} is a fluoroalkylene group having 3 carbon atoms,
R^{f4} is a fluoroalkylene group having 4 carbon atoms,
R^{f5} is a fluoroalkylene group having 5 carbon atoms,
R^{f6} is a fluoroalkylene group having 6 carbon atoms,
R^{f7} is a fluoroalkylene group having 1 to 6 carbon atoms,
m11, m12, m13, m14, m15, and m16 each independently represent an integer of 0 or 1 or more, m17 is an integer of 0 or 1, and
m11+m12+m13+m14+m15+m16+m17 is an integer of 0 to 200.

Note that the bonding order of (R^{f1}O) to (R^{f6}O) in the formula (G1-1) is random.
m11 to m16 in the formula (G1-1) represent the number of (R^{f1}O) to (R^{f6}O), respectively, and do not represent arrangement. For example, (R^{f5}O)ₘ₅ represents that the number of (R^{f5}O ) is m5, and does not represent a block arrangement structure of (R^{f5}O)ₘ₅. Similarly, the order of description of (R^{f1}O) to (R^{f6}O) does not represent the bonding order of the respective units.

When m17 is 0, the terminal of G¹ bonded to CH₂ is —O—. When m17 is 1, the terminal of G¹ bonded to CH₂ is a carbon atom (carbon atom at the terminal of R^{f7}).

The fluoroalkylene group having 3 to 6 carbon atoms may be a linear fluoroalkylene group or a fluoroalkylene group having a branched or ring structure.

For example, when the compound (A1) is used as a raw material of a surface treatment agent, m11+m12+m13+m14+m15+m16 is an integer of 1 to 200, that is, G¹ is preferably a polyoxyfluoroalkylene chain from the viewpoint of water/oil repellency, fingerprint removability, and the like.

In addition, for example, from the viewpoint of chemical stability of the compound (A1), m17 is preferably 1.

Specific examples of R^{f1} include —CF₂— and —CHF—.

Specific examples of R^{f2} include —CF₂CF₂—, —CHFCF₂—, —CHFCHF—, —CH₂CF₂—, and —CH₂CHF—.

Specific examples of R^{f3} include —CF₂CF₂CF₂—, —CF₂CHFCF₂—, —CF₂CH₂CF₂—, —CHFCF₂CF₂—, —CHFCHFCF₂—, —CHFCHFCHF—, —CHFCH₂CF₂—, —CHFCH₂CF₂—, —CH₂CF₂CF₂—, —CH₂CHFCF₂—, —CH₂CH₂CF₂—, —CH₂CF₂CHF—, —CH₂CHFCHF—, —CH₂CH₂CHF—, —CF(CF₃)—CF₂—, —CF(CHF₂)—CF₂—, —CF(CH₂F)—CF₂—, —CF(CH₃)—CF₂—, —CF(CF₃)—CHF—, —CF(CHF₂)—CHF—, —CF(CH₂F)—CHF—, —CF(CH₃)—CHF—, —CF(CF₃)—CH₂—, —CF(CHF₂)—CH₂—, —CF(CH₂F)—CH₂—, —CF(CH₃)—CH₂—, —CH(CF₃)—CF₂—, —CH(CHF₂)—CF₂—, —CH(CH₂F)—CF₂—, —CH(CH₃)—CF₂—, —CH(CF₃)—CHF—, —CH(CHF₂)—CHF—, —CH(CH₂F)—CHF—, —CH(CH₃)—CHF—, —CH(CF₃)—CH₂—, —CH(CHF₂)—CH₂—, and —CH(CH₂F)—CH₂—.

Specific examples of R^{f4} include —CF₂CF₂CF₂CF₂—, —CHFCF₂CF₂CF₂—, —CH₂CF₂CF₂CF₂—, —CF₂CHFCF₂CF₂—, —CHFCHFCF₂CF₂—, —CH₂CHFCF₂CF₂—, —CF₂CH₂CF₂CF₂—, —CHFCH₂CF₂CF₂—, —CH₂CH₂CF₂CF₂—, —CHFCF₂CHFCF₂—, —CH₂CF₂CHFCF₂—, —CF₂CHFCHFCF₂—, —CHFCHFCHFCF₂—, —CH₂CHFCHFCF₂—, —CF₂CH₂CHFCF₂—, —CHFCH₂CHFCF₂—, —CH₂CH₂CHFCF₂—, —CF₂CH₂CH₂CF₂—, —CHFCH₂CH₂CF₂—, —CH₂CH₂CH₂CF₂—, —CHFCH₂CH₂CHF—, —CH₂CH₂CH₂CHF—, and -cycloC₄F₆—.

Specific examples of R^{f5} include —CF₂CF₂CF₂CF₂CF₂—, —CHFCF₂CF₂CF₂CF₂—, —CH₂CHFCF₂CF₂CF₂—, —CF₂CHFCF₂CF₂CF₂—, —CHFCHFCF₂CF₂CF₂—, —CH₂CHFCF₂CF₂CF₂—, —CF₂CH₂CF₂CF₂CF₂—, —CHFCH₂CF₂CF₂CF₂—, —CH₂CH₂CF₂CF₂CF₂—, —CF₂CF₂CHFCF₂CF₂—, —CHFCF₂CHFCF₂CF₂—, —CH₂CF₂CHFCF₂CF₂—, —CF₂CF₂CHFCF₂CF₂—, —CHFCF₂CHFCF₂CF₂—, —CH₂CF₂CHFCF₂CF₂—, —CH₂CF₂CF₂CF₂CH₂—, and —cycloC₅F₈—.

Specific examples of R^{f6} include —CF₂CF₂CF₂CF₂CF₂CF₂—, —CF₂CF₂CHFCHFCF₂CF₂—, —CHFCF₂CF₂CF₂CF₂CF₂—, —CHFCHFCHFCHFCHFCHF—, —CHFCF₂CF₂CF₂CF₂CH₂—, —CH₂CF₂CF₂CF₂CF₂CH₂—, and -cycloC₆F₁₀—.

In addition, specific examples of R^{f0} and R^{f7} include the same ones as those mentioned in the above R^{f1} to R^{f6}, and R^{f7} is preferably perfluoroalkyl group.

Here, -cycloC₄F₆— means a perfluorocyclobutanediyl group, and specific examples thereof include a perfluorocyclobutane-1,2-diyl group. -cycloC₅F₈— means a perfluorocyclopentanediyl group, and specific examples thereof include a perfluorocyclopentane-1,3-diyl group. -cycloC₆F₁₀— means a perfluorocyclohexanediyl group, and specific examples thereof include a perfluorocyclohexane-1,4-diyl group.

When the compound (A1) is used as a raw material of a surface treatment agent, G¹ is preferably a monovalent group having a (poly)oxyfluoroalkylene chain, and more preferably a monovalent group having a polyoxyfluoroalkylene chain, from the viewpoint of further excellent water/oil repellency, friction resistance, and fingerprint dirt removability. Among them, it is preferable to have structures represented by the following formulae (F1) to (F3).

(R^{f1}O)ₘ₁₁—(R^{f2}O)ₘ₁₂—(R^{f7})ₘ₁₇ Formula (F1)

(R^{f2}O)ₘ₁₂—(R^{f4}O)ₘ₁₄—(R^{f7})ₘ₁₇ Formula (F2)

(R^{f3}o)ₘ₁₃—(R^{f7})ₘ₁₇ Formula (F3)
where, each reference sign of the formulae (F1) to (F3) is the same as those in the formula (G1-1).

In the above formulae (F1) and (F2), the bonding order of (R^{f1}O) and (R^{f2}O), and (R^{f2}O) and (R^{f4}O) is random. For example, (R^{f1}O) and (R^{f2}O) may be alternately arranged, (R^{f1}O) and (R^{f2}O) may be each arranged in a block, or may be random. The same applies to the formula (F3).

In the formula (F1), m11 is preferably 1 to 30, and more preferably 1 to 20. In addition, m12 is preferably 1 to 30, and more preferably 1 to 20.

In the formula (F2), m12 is preferably 1 to 30, and more preferably 1 to 20. In addition, m14 is preferably 1 to 30, and more preferably 1 to 20.

In the formula (F3), m13 is preferably 1 to 30, and more preferably 1 to 20.

The fluoroalkylene group in G² may be linear, or may have a branched or ring structure. The number of carbon atoms in the fluoroalkylene group is preferably 1 to 30, more preferably 1 to 20, still more preferably 1 to 10, and particularly preferably 1 to 6 from the viewpoint of increasing the yield of the present production method.

Specific examples of the fluoroalkylene group include the same groups as those mentioned in the above R^{f1} to R^{f6}.

The divalent group having a (poly)oxyfluoroalkylene chain in G² is a fluoroalkylene group having —O— at two terminals bonded to CH₂ each independently, or —O— between carbon-carbon atoms of a carbon chain having 2 or more carbon atoms, or is a combination thereof in the formula (A2). From the viewpoint of ease of production and the like, G 2 preferably has a structure represented by the following formula (G2-1).

—(O)ₘ₁₀—[(R^{f1}O)ₘ₁₁(R^{f2}O)ₘ₁₂(R^{f3}O)ₘ₁₃(R^{f4}O)ₘ₁₄(R^{f5}O)ₘ₁₅(R^{f6}O)ₘ₁₆]—(R^{f7})ₘ₁₇— Formula (G2-1)
where, m10 is an integer of 0 or 1, and R^{f1}, R^{f2}, R^{f3}, R^{f4}, R^{f5}, R^{f6}, R^{f7}, m11, m12, m13, m14, m15, m16, and m17 are the same as those in the G¹. Note that the bonding order of (R^{f1}O) to (R^{f6}O) in the formula (G2-1) is random and is as described in the above formula (G1-1).

When m17 is 0, one terminal of G² bonded to CH₂ is —O—. When m17 is 1, one terminal of G² bonded to CH₂ is a carbon atom (carbon atom at the terminal of R^{f7}). In addition, when m10 is 1, one terminal of G² bonded to CH₂ is —O—. When m10 is 0, one terminal of G² bonded to CH₂ is a carbon atom (carbon atom at any terminal of R^{f1} to R^{f7}). Note that m10 and m17 are each independently 0 or 1.

For example, when the compound (A2) is used as a raw material of a surface treatment agent, m10+m11+m12+m13+m14+m15+m16 is an integer of 2 to 200, that is, G² is preferably a polyoxyfluoroalkylene chain from the viewpoint of water/oil repellency, fingerprint removability, and the like.

In addition, for example, from the viewpoint of chemical stability of the compound (A2), it is preferable that m10 be 0 or m17 be 1, and it is more preferable that m10 be 0 and m17 be 1.

When the obtained compound (A2) is used as a surface treatment agent or a raw material thereof, G² preferably has a structure represented by the following formulae (F4) to (F6) from the viewpoint of further excellent water/oil repellency, friction resistance, and fingerprint dirt removability.

—(O)ₘ₁₀—(R^{f1}O)ₘ₁₁—(R^{f2}O)ₘ₁₂—(R^{f}7)ₘ₁₇ Formula (F4)

—(O)ₘ₁₀—(R^{f2}O)ₘ₁₂—(R^{f4}O)ₘ₁₄—(R^{f}7)ₘ₁₇ Formula (F5)

—(O)ₘ₁₀—(R^{f3}O)ₘ₁₃—(R^{f7})ₘ₁₇ Formula (F6)
where, each reference sign of the formulae (F4) to (F6) is the same as those in the formula (G2-1).

In the above formulae (F4) and (F5), the bonding order of (R^{f1}O) and (R^{f2}O), and (R^{f2}O) and (R^{f4}O) is random. For example, (R^{f1}O) and (R^{f2}O) may be alternately arranged, (R^{f1}O) and (R^{f2}O) may be each arranged in a block, or may be random.

In the formula (F4), mll is preferably 1 to 30, and more preferably 1 to 20.In addition, m12 is preferably 1 to 30, and more preferably 1 to 20.

In the formula (F5), m12 is preferably 1 to 30, and more preferably 1 to 20. In addition, m14 is preferably 1 to 30, and more preferably 1 to 20.

In the formula (F6), m13 is preferably 1 to 30, and more preferably 1 to 20.

The ratio of fluorine atoms in the fluoroalkyl chain and the (poly)oxyfluoroalkylene chain [{number of fluorine atoms/(number of fluorine atoms+number of hydrogen atoms)}×100 (%)] in G¹ and G² is preferably 40% or more, more preferably 50% or more, and still more preferably 60% or more from the viewpoint of excellent water/oil repellency and fingerprint removability.

In addition, the molecular weight of the (poly)oxyfluoroalkylene chain part is preferably 200 to 30,000, more preferably 600 to 25,000, still more preferably 800 to 20,000, and particularly preferably 1,000 to 8,000 from the viewpoint of wear resistance.
X¹ is a halogen atom. Examples of the halogen atom include F, Cl, Br, and I. In the production method described later, when a substituent is introduced into X¹, X¹ is preferably Cl, Br or I, more preferably Br or I, and still more preferably I.
M represents a boron-based substituent BR¹R², and R² are each independently a group represented by —OH, —R¹¹, —OR¹¹, —O—C(O)—R¹¹, or —NR¹¹R¹², R¹¹ is a hydrocarbon group which may have a substituent or a heteroatom in the carbon chain, two R¹¹'s may be linked to form a ring structure, and R⁶ is a hydrogen atom or an alkyl group having 1 to 6 carbon atoms.

Examples of the hydrocarbon group in R¹¹ include an aliphatic hydrocarbon group (linear alkyl group, branched alkyl group, cycloalkyl group, and the like). The aliphatic hydrocarbon group may have a double bond or a triple bond in the carbon chain. Examples of the substituent that the hydrocarbon group may have include a halogen atom, a hydroxy group, an amino group, a nitro group, a sulfo group, and an oxo group, from the viewpoint of the stability of the compound in the present production method, a halogen atom is preferable. Examples of the halogen atom include a fluorine atom, a chlorine atom, and a bromine atom.

Heteroatoms may be included in the carbon chain alone or in combination with other substituents such as an oxo group. Specific examples of the partial structure containing a heteroatom include —C(O)NR²⁵—, —C(O)O—, —C(O)—, —O—, —NR²⁵—, —S—, —OC(O)O—, —NHC(O)O—, —NHC(O)NR²⁵—, —SO₂NR²⁵—, —Si(R²⁵)₂—, and —Osi(R²⁵)₂—. Here, R²⁵ is a hydrogen atom, an alkyl group having 1 to 6 carbon atoms, or a phenyl group.

Examples of the structure in which two R¹¹'s are linked to form a ring structure include *—R¹¹—*, —O—R¹¹—*, *—O—R¹¹—O—*, *—I—C(O)—R¹¹—C(O)—O—*, and *—NR¹²—R¹¹—NR¹²—*. Here, * is a linking group bonded to B, and a ring structure containing B is formed.

Specific examples of BR¹R² include the following.

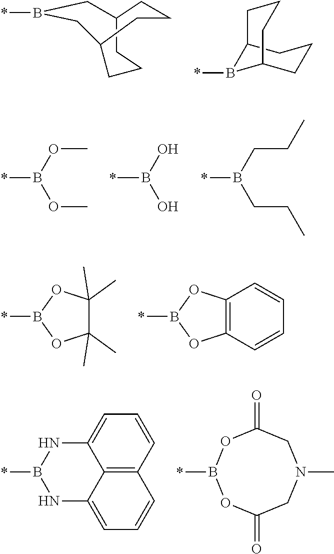

Specific examples of the compound (A1) and the compound (A2) include the following compounds.

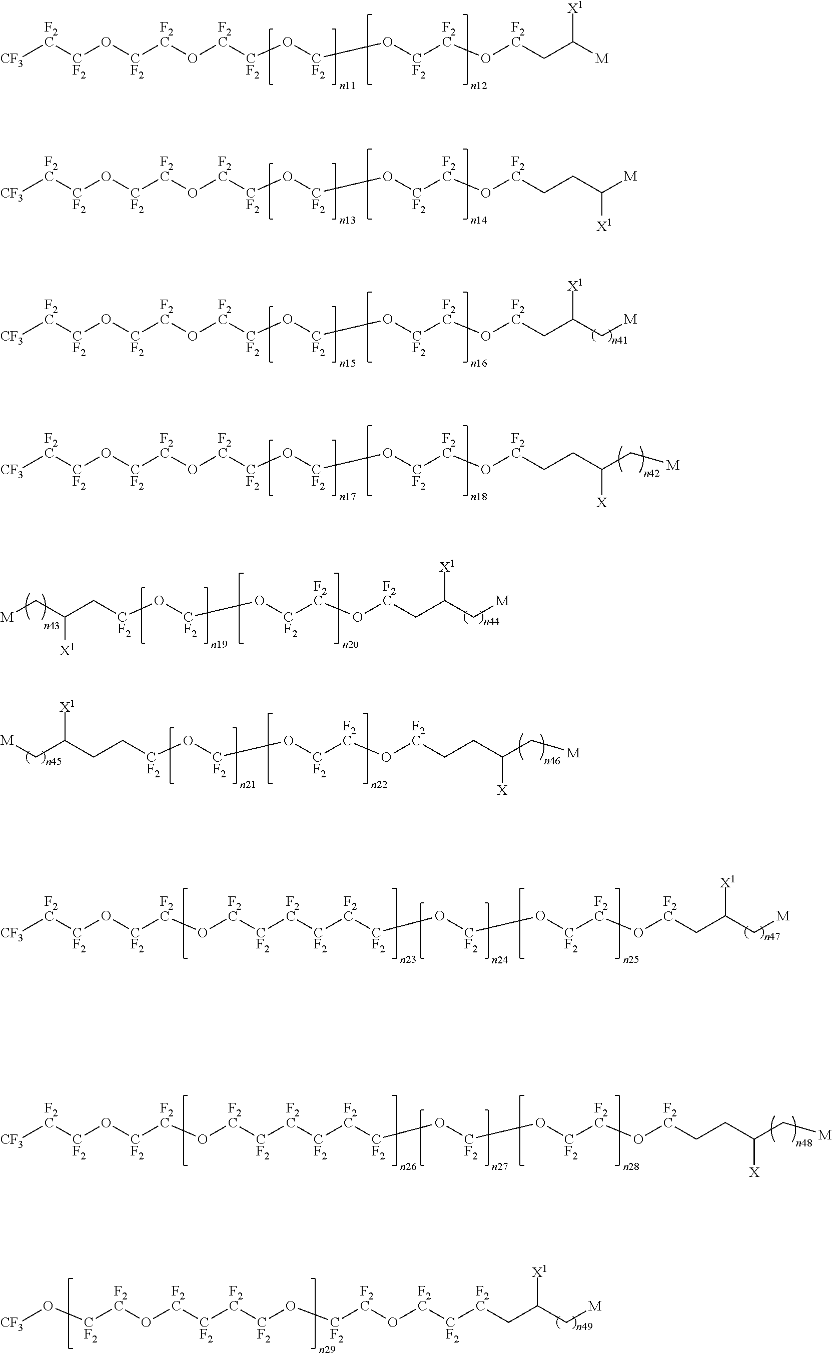

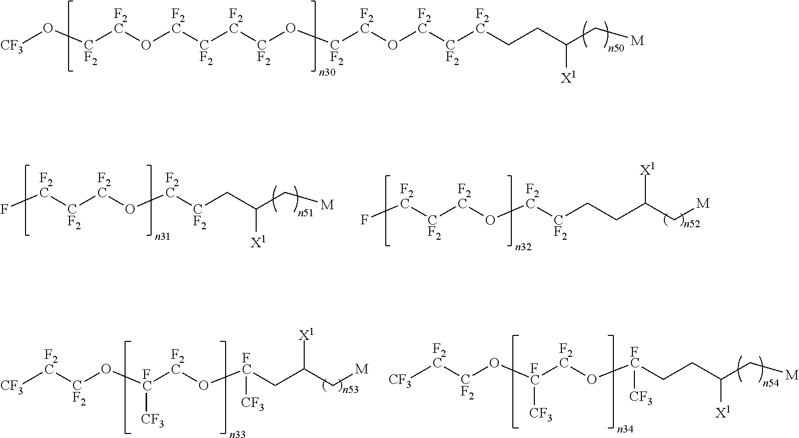

where X¹ and M are as described above, n11 to n34 represent the number of repeating units and are each independently an integer of 1 to 200, n41 and n42 are each independently an integer of 1 to 20, and n43 to n54 are each independently an integer of 0 to 20.

### Method for Producing Compound (A1) or Compound (A2)

The method for producing the present compound is not particularly limited, but the following production method is preferable from the viewpoint of relatively easy availability of raw materials and excellent yield.

That is, the method for producing the compound (A1) or the compound (A2) according to the present invention includes reacting a compound represented by the following formula (E1) or (E2) with a compound represented by the following formula (H1).

G¹-(CH₂)ₘ₄—X¹ Formula (E1)

X¹—(CH₂)ₘ₅-G²-(CH₂)ₘ₆—X¹ Formula (E2)

CH₂′CH—(CH₂)ₙ-M Formula (H1)
where, in the formula,
G¹, G², X, and M are the same as those in the formula (A1) or (A2), and preferred embodiments are also the same.
n is an integer of 0 to 20 and corresponds to n1, n2, or n3 in the formula (A1) or the compound (A2).

In addition, m4, m5, and m6 are each independently 0 or 1, and m4+1, m5+1, and m6+1 correspond to m1, m2, or m3 in the formula (A1) or the compound (A2).

For example, the compound (E1) or the compound (E2), the compound (H1), and as necessary, a radical polymerization initiator such as an azo-based polymerization initiator are added to a solvent and heated, and accordingly, the compound (E1) or the compound (E2) is added to the olefin of the compound (H1), and the compound (A1) or the compound (A2) can be obtained. The reaction temperature is not particularly limited, and may be, for example, 40 to 120° C. or the like. In addition, the reaction time is appropriately adjusted according to the amount of the compound and the like, and may be, for example, 1 to 40 hours.

Specific examples of the compound (E1) and the compound (E2) include the following compounds.

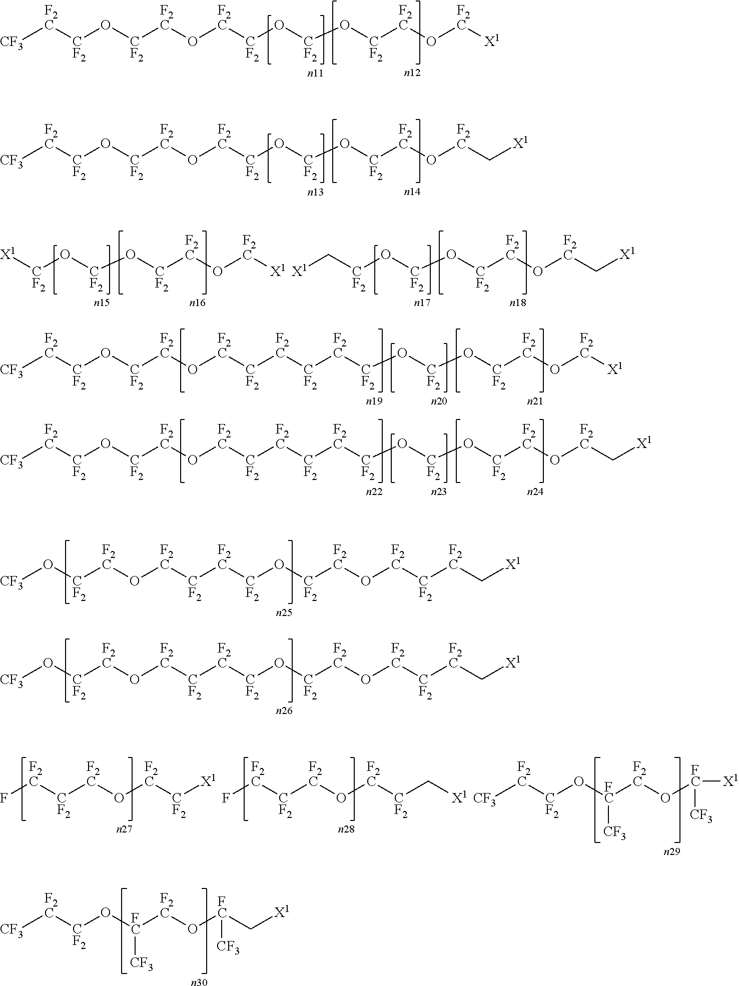

where, X¹ is as described above, and n11 to n30 represent the number of repeating units, and each independently represent an integer of 1 to 200.

Specific examples of the compound (H1) include the following compounds.

CH₂═CH—BR¹R², CH₂═CH—CH₂—CR¹R², CH₂═CGH—CH₂CH₂—CR¹R², CH₂═CH—CH₂CH₂CH₂—BR¹R², CH₂═CH—CH₂CH₂CH₂CH₂—BR¹R², CH₂═CH—CH₂CH₂CH₂CH₂CH₂—BR¹R² where, BR¹R² is as described above.

The compound (E1) and the compound (E2) can be produced, for example, by a method in which compounds represented by the following formulae (E1-2) and (E2-2) are reacted with a halomethane such as iodomethane in the presence of triphenylphosphine to halogenate the compounds, a method in which the compounds are reacted with a halogen molecule in the presence of triphenylphosphine to halogenate the compounds, or the like. In addition, a commercially available product having a desired structure may be used.

G¹OH Formula (E1-2)

HO-G²-OH Formula (E2-2)
where, each reference sign in the formula is as described above.

In addition, the compound (H1) may be synthesized, or a commercially available product or the like may be used.

### Method for Producing Compound (B1) or Compound (B2)

In the method for producing a compound represented by the following formula (B1) or (B2) according to the present invention, M in the compound (A1) or the compound (A2) is converted into hydrogen or a hydrocarbon group.

G¹-(CH₂)ₘ₁—CHX¹—(CH₂)ₙ₁—R³ Formula (B1)

R³—(CH₂)ₙ₂—CHX¹—(CH₂)ₘ₂-G²-(CH₂)ₘ₃—CHX¹—(CH₂)ₙ₃—R³ Formula (B2)

However, in the formula, G¹, G², X¹, n1, n2, n3, m1, m2, and m3 are the same as those in the compound (A1) or the compound (A2), and preferred embodiments are also the same.

R³ is a hydrogen atom or a hydrocarbon group which may have a substituent or a heteroatom.

Examples of the hydrocarbon group in R³ include an aliphatic hydrocarbon group (linear alkyl group, branched alkyl group, cycloalkyl group, and the like), an aromatic hydrocarbon group (phenyl group and the like), and a group consisting of combinations thereof. The aliphatic hydrocarbon group may have a double bond or a triple bond in the carbon chain. Examples of the combination include a group in which an alkylene group and an aryl group are directly linked via a heteroatom.

Examples of the substituent that the hydrocarbon group may have include a halogen atom, a hydroxy group, an amino group, a nitro group, a sulfo group, and an oxo group, from the viewpoint of the stability of the compound in the present production method, a halogen atom is preferable. Examples of the halogen atom include a fluorine atom, a chlorine atom, and a bromine atom.

Heteroatoms may be included in the carbon chain alone or in combination with other substituents such as an oxo group. Specific examples of the partial structure containing the heteroatom include —C(O)NR²⁶—, —C(O)O—, —C(O)—, —O—, —NR²⁶—, —S—, —OC(O)O—, —NHC(O)O—, —NHC(O)NR²⁶—, —SO₂NR²⁶—, —Si(R²⁶)₂—, —OSi(R²⁶)₂—, —Si(CH₃)₂—Ph—Si(CH₃)₂—, and a divalent organopolysiloxane residue. Here, R²⁶ is a hydrogen atom, an alkyl group having 1 to 6 carbon atoms, or a phenyl group, and Ph is a phenylene group. From the viewpoint of ease of production of the present compound, the number of carbon atoms in the alkyl group of R²⁶ is preferably 1 to 3, and particularly preferably 1 and 2.

When another substituent is further introduced into the compound (B1) or the compound (B2), the hydrocarbon group in R³ preferably has a carbon-carbon double bond. When the compound (B1) or the compound (B2) has a double bond, another substituent can be easily introduced by an addition reaction.

Examples of the method for producing the compound (B1) or the compound (B2) include a method of reacting the compound (A1) or the compound (A2) with a compound represented by the following formula (C1).

R³—X² Formula (C1)
where, in the formula,
R³ is as described above, and
X² is a halogen atom.

From the viewpoint of reactivity, the halogen atom in X 2 is preferably a chlorine atom, a bromine atom, or an iodine atom, more preferably a chlorine atom or a bromine atom, and still more preferably a bromine atom.

Preferred specific examples of the compound (C1) include the following compounds. In addition, X² is as described above.

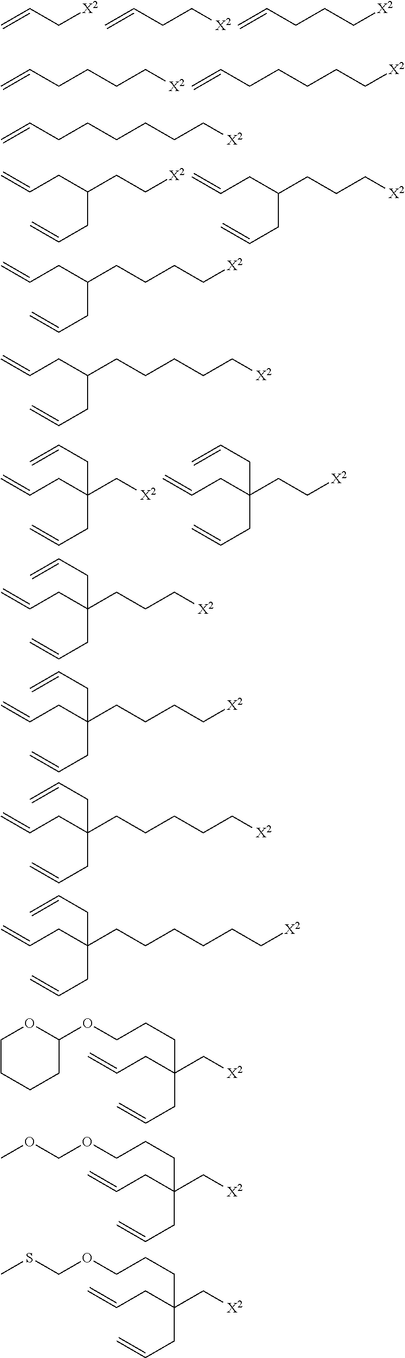

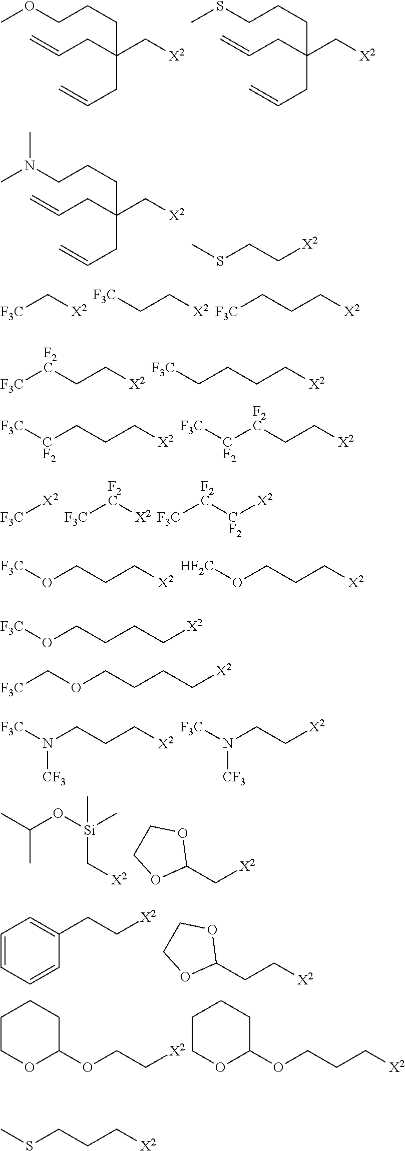

The compound (A1) or the compound (A2) and the compound represented by the following formula (C1) can be reacted, for example, by heating in a solvent in the presence of a catalyst and a base.

Examples of the catalyst include known metal catalysts, and among them, transition metal compounds are preferable, and among them, compounds containing elements from groups 8 to 11 are more preferable, and compounds containing one or more elements selected from copper, nickel, palladium, and cobalt are preferable.

In the metal catalyst containing copper, the copper may be any of zerovalent, monovalent, divalent, and trivalent, and among them, monovalent or divalent copper salts or complex salts are preferable from the viewpoint of catalytic ability. Further, copper chloride is more preferable from the viewpoint of easy availability and the like. As copper chloride, either CuCl or CuCl₂ can be suitably used. In addition, copper chloride may be an anhydride or a hydrate, but copper chloride anhydride is more preferable from the viewpoint of catalytic ability.

In the metal catalyst containing nickel, the nickel may be any of zerovalent, monovalent, divalent, and trivalent, and among them, monovalent or divalent nickel salts or complex salts are preferable from the viewpoint of catalytic ability. Further, nickel chloride (NiCl₂) is more preferable from the viewpoint of easy availability and the like. In addition, nickel chloride may be an anhydride or a hydrate, but nickel chloride anhydride is more preferable from the viewpoint of catalytic ability.

In the metal catalyst containing palladium, the palladium may be any of a zerovalent compound, a monovalent compound, a divalent compound, and a trivalent compound, and among them, zerovalent or divalent palladium salts or complex salts are preferable from the viewpoint of catalytic ability. Furthermore, tris(dibenzylideneacetone)dipalladium (Pd₂(dba)₃) and palladium acetate (Pd(OAc)₂) are more preferable from the viewpoint of easy availability and the like. In addition, tris(dibenzylideneacetone)dipalladium and palladium acetate may be anhydrides or hydrates, but tris(dibenzylideneacetone)dipalladium anhydride and palladium acetate anhydride are more preferable from the viewpoint of catalytic ability.

In the reaction of the present production method, examples of the above ligand that may be used in combination with the metal catalyst as necessary include 1,3-butadiene, tricyclohexylphosphine, 1,1-bis(diphenylphosphino)ferrocene phenylpropyne, and tetramethylethylenediamine (TMEDA).

The base is preferably a strong base having low nucleophilicity, and examples thereof include potassium tert-butoxide, lithium diisopropylamide, potassium hexamethyldisilazide, and lithium-2,2,6,6-tetramethylpiperide.

In addition, the solvent may be appropriately selected from solvents inert to the present reaction. Examples thereof include ether-based solvents such as diethyl ether, tetrahydrofuran (THF), and dioxane. In addition, when the compound (A1) and the compound (A2) are compounds having a relatively high fluorine atom content, the fluorine-based solvent is preferably used alone or in combination with the solvent.

Examples of the fluorine-based solvent include hydrofluorocarbons (1H,4H-perfluorobutane, 1H-perfluorohexane, 1,1,1,3,3-pentafluorobutane, 1,1,2,2,3,3,4-heptafluorocyclopentane, 2H,3H-perfluoropentane, and the like), hydrochlorofluorocarbons (3,3-dichloro-1,1,1,2,2-pentafluoropropane, 1,3-dichloro-1,1,2,2,3-pentafluoropropane (HCFC-225cb), and the like), hydrofluoroethers (CF₃CH₂OCF₂CF₂H(AE-3000), (perfluorobutoxy)methane, (perfluorobutoxy)ethane, and the like), hydrochlorofluoroolefins ((Z)-1-chloro-2,3,3,4,4,5,5-heptafluoro-l-pentene (HCFO-1437 dycc (Z) form), (E)-1-chloro-2,3,3,4,4,5,5-heptafluoro-l-pentene (HCFO-1437 dycc (E) form), (Z)-1-chloro-2,3,3-trifluoro-1-propene (HCFO-1233 yd (Z) form), (E)-1-chloro-2,3,3-trifluoro-1-propene (HCFO-1233 yd (E) form), and the like), and fluorine-containing aromatic compounds (perfluorobenzene, m-bis(trifluoromethyl)benzene(SR-solvent), p-bis(trifluoromethyl)benzene, and the like).

### Method for Producing Compound (B3) or Compound (B4)

In the method for producing a compound represented by the following formula (B3) or (B4) according to the present invention, X¹ in the compound (A1) or the compound (A2) is converted into hydrogen or a hydrocarbon group.

G¹CH₂—CHR³—(CH₂)ₙ₁-M Formula (B3)

M-(CH₂)ₙ₂—CHR³—CH₂-G²-CH₂—CHR³—(CH₂)ₙ₃-M Formula (B4)
where, in the formula, G¹, G², M, R³, n1, n2, and n3 are as described above, and preferred embodiments are also the same.

Examples of the method for producing the compound (B3) or the compound (B4) include a method of reacting the compound (A1) or the compound (A2) with a compound represented by the following formula (C2).

R³—MgR⁴ Formula (C2)
where, in the formula, R³ is as described above, and
R⁴ is a halogen atom or a hydrocarbon group which may have a substituent or a heteroatom.

From the viewpoint of reactivity, the halogen atom in R⁴ is preferably a chlorine atom, a bromine atom, or an iodine atom, more preferably a chlorine atom or a bromine atom, and still more preferably a bromine atom.

Examples of the hydrocarbon group in R⁴ include those similar to those in the R³. In addition, when R⁴ is a hydrocarbon group, R⁴ may be introduced instead of R³ in the reaction in the present production method, and for example, the following compounds (B5) to (B7) and the like may be generated.

G¹-(CH₂)ₘ₁—CHR⁴—(CH₂)ₙ₁-M Formula (B5)

M-(CH₂)ₙ₂—CHR³—(CH₂)ₘ₂-G²-(CH₂)ₘ₃—CHR⁴—(CH₂)ₙ₃-M Formula (B6)

M-(CH₂)ₙ₂—CHR⁴—(CH₂)ₘ₂-G²-(CH₂)ₘ₃—CHR⁴—(CH₂)ₙ₃-M Formula (B7)
where, each reference sign in the formula is as described above.

By having R³ and R⁴ as substituents having the same structure, the compounds (B5) to (B7) as by-products are the same compounds as the compound (B1) or (B2).

When R⁴ is a substituent lower in reactivity than R³, generation of the compounds (B5) to (B7) as by-products can be suppressed.

In addition, when the compounds (B5) to (B7) are produced, separation by column chromatography or the like may be performed as necessary, and a mixture containing the compounds (B5) to (B7) may be used as it is depending on the application of the compounds (B5) to (B7).

Preferred specific examples of the compound (C2) include the following compounds. In addition, R⁴ is as described above.

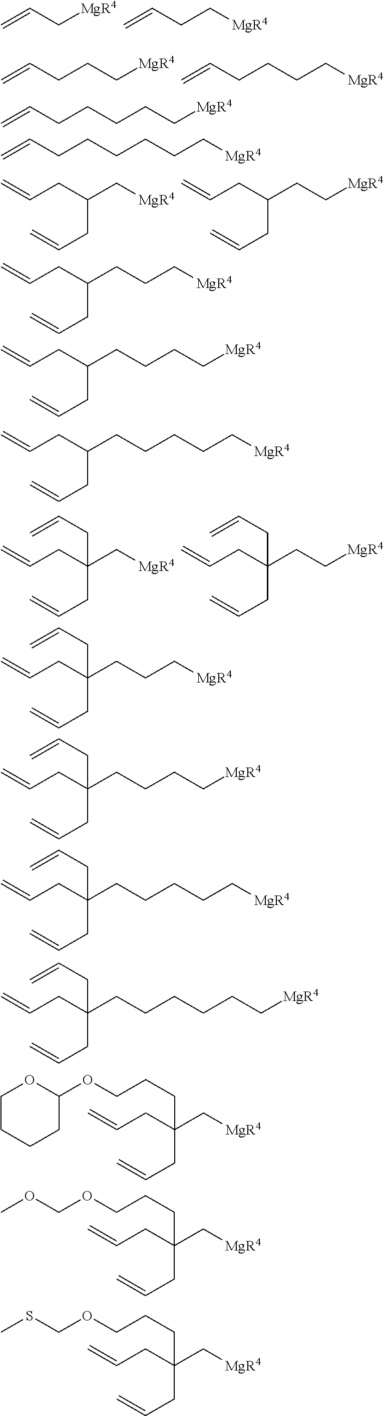

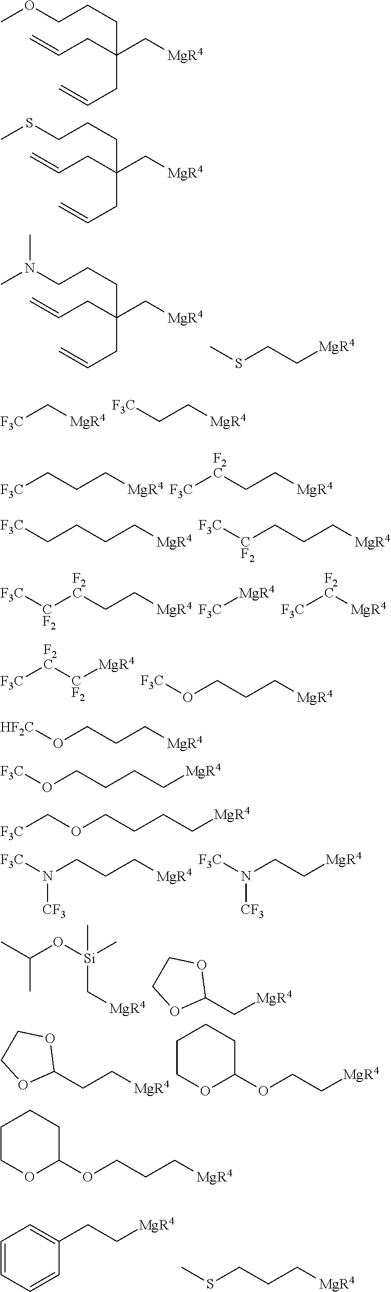

The compound (A1) or the compound (A2) and the compound represented by the following formula (C2) can be reacted, for example, by heating in the solvent, and a catalyst and a base may be added as necessary. Examples of the solvent, catalyst, and base include those similar to those described above.

### Method for Producing Surface Treatment Agent

In the method for producing a surface treatment agent according to the present invention, a reactive silyl group is introduced into the compound (B1), the compound (B2), the compound (B3), or the compound (B4) obtained by the above-described production method. The compound having a (poly)oxyfluoroalkylene chain and a hydrolyzable silyl group can form a surface layer exhibiting high lubricity, water/oil repellency, and the like on the surface of the substrate, and thus is suitably used as a surface treatment agent.

The method for introducing a reactive silyl group into the compound (B1) to the compound (B4) may be appropriately selected according to the substituent of the compound (B1) to the compound (B4). As an example, when the compound (B1) to the compound (B4) have a double bond, the compound (B1) to the compound (B4) can be introduced by subjecting the double bond and the following compound (J1) or (J2) to a hydrosilylation reaction.

HSi(R⁴⁰)_{3-c}(L)_{c} Formula (J1)

HSi(R⁴¹)₃₋ₖ[—(OSi(R⁴²)₂)ₚ—O-Si(R⁴⁰)_{3-c}(L)_{c}]ₖ Formula (J2)
where, in the formula,
R⁴⁰ is an alkyl group, and when there are a plurality of R⁴⁰'s, the R⁴⁰'s may be the same as or different from each other,
L is a hydrolyzable group or a hydroxyl group, and a plurality of L's may be the same as or different from each other,
R⁴¹ is an alkyl group, and when there are a plurality of R⁴¹'s, the R⁴¹'s may be the same as or different from each other,
R⁴² is an alkyl group, a phenyl group, or an alkoxy group, and two R⁴²'s may be the same or different;
c is 2 or 3,
k is 2 or 3, and
p is an integer of 0 to 5, and when p is two or more, two or more (OSi(R⁴²)₂)'s may be the same as or different from each other.

In addition, the compound (J1) may be synthesized, or a commercially available product may be used. In addition, the compound (J2) can be produced, for example, by the method described in International Patent Publication No. WO 2019/208503.

The reactive silyl group is a group in which one or both of a hydrolyzable group and a hydroxyl group are bonded to a silicon atom. The hydrolyzable group is a group that becomes a hydroxyl group by a hydrolysis reaction. That is, the hydrolyzable silyl group becomes a silanol group (Si—OH) by a hydrolysis reaction.

The silanol group further undergoes a dehydration condensation reaction between molecules to form a Si—O—Si bond. In addition, the silanol group undergoes a dehydration condensation reaction with a hydroxyl group (substrate —OH) on the surface of the substrate to form a chemical bond (substrate —O—Si).

Examples of the hydrolyzable group include an alkoxy group, a halogen atom, an acyl group, and an isocyanate group. The alkoxy group is preferably an alkoxy group having 1 to 6 carbon atoms.

The halogen atom is preferably a chlorine atom.

The hydrolyzable group is preferably an alkoxy group or a halogen atom from the viewpoint of ease of production. As the hydrolyzable group, an alkoxy group having 1 to 4 carbon atoms is preferable from the viewpoint of having less outgassing at the time of coating and being excellent in storage stability of the present compound, an ethoxy group is particularly preferable when long-term storage stability of the present compound is required, and a methoxy group is particularly preferable when the reaction time after coating the substrate with the surface treatment agent is shortened.

According to the above production method, for example, a surface treatment agent represented by the following formula is obtained.

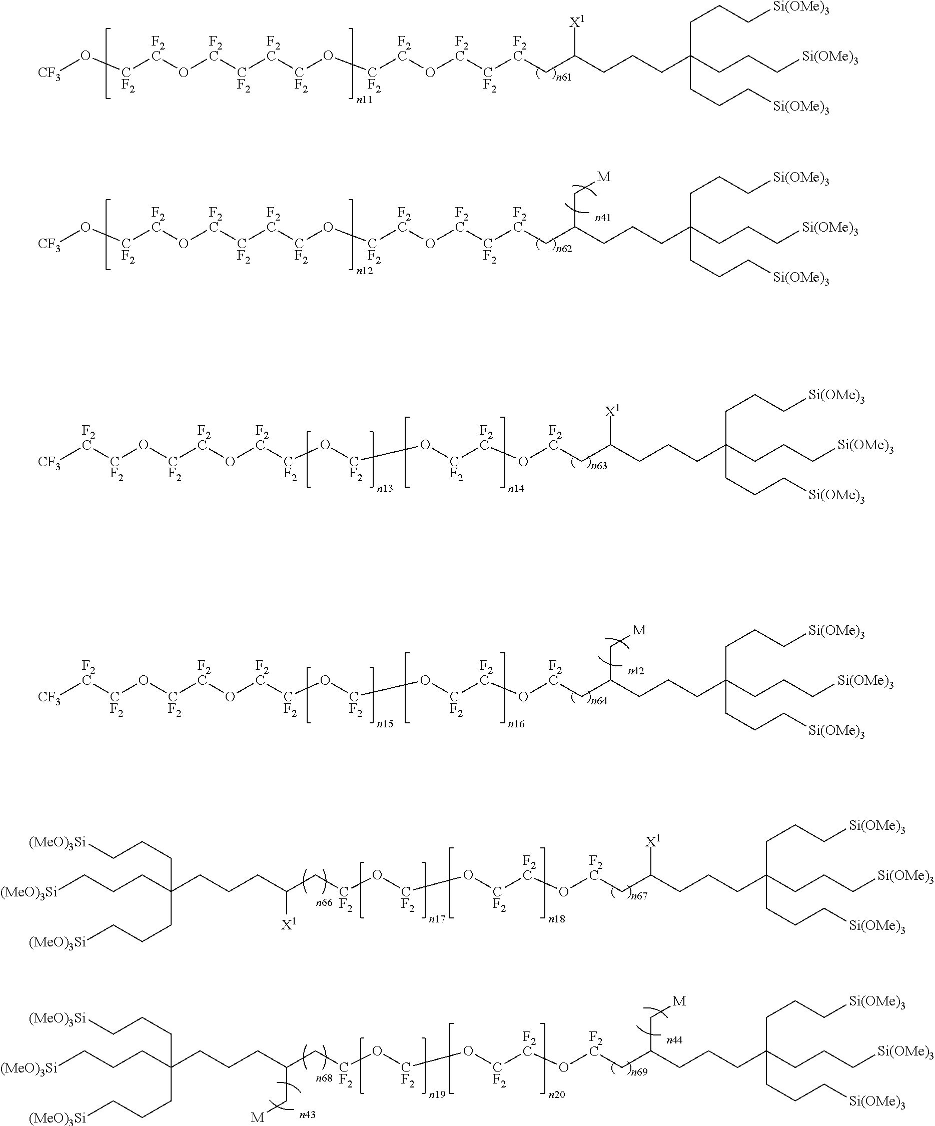

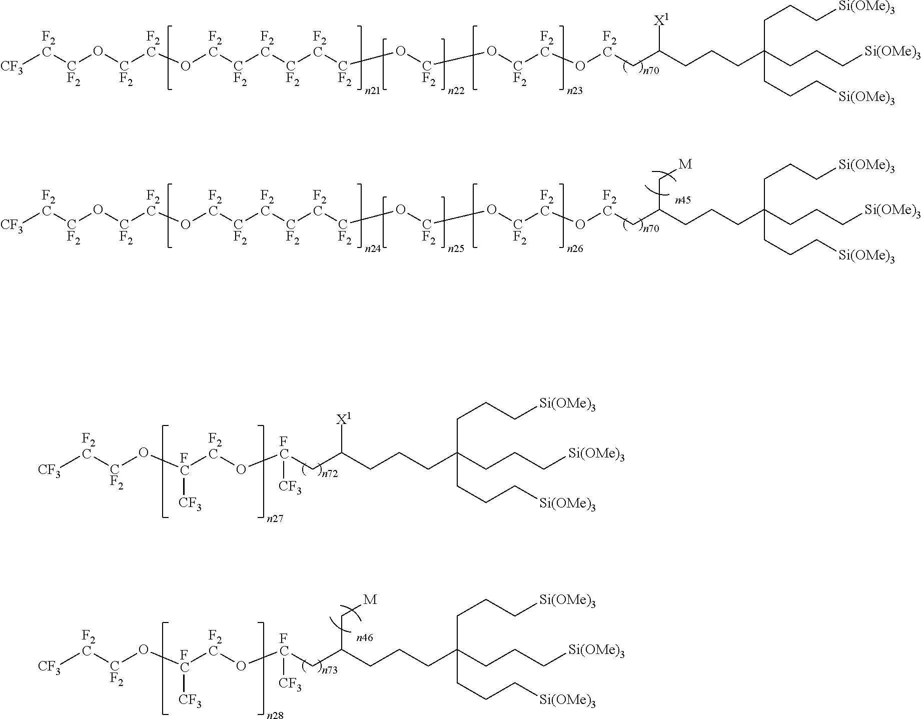

However, X¹ and M are as described above, n11 to n28 are each independently an integer of 1 to 200, n41 to n46 are each independently an integer of 1 to 20, and n61 to n73 are each independently 1 or 2.

In addition, M in the above formula may be removed according to the application of the surface treatment agent or the like. M can be removed, for example, by the action of an acid in the presence of a Lewis base.

Examples of the substrate to which the surface treatment agent is applied include substrates required to be imparted with water/oil repellency. Examples thereof include other articles (for example, stylus), substrates that may be used by being brought into contact with human fingers, substrates that may be held by human fingers during operation, and substrates that may be placed on other articles (for example, placing tables).

Examples of the material of the substrate include metal, resin, glass, sapphire, ceramic, stone, and composite materials thereof. The glass may be chemically strengthened. A base film such as a SiO2 film may be formed on the surface of the substrate.

As the substrate, a substrate for a touch panel, a substrate for a display, and a spectacle lens are preferred, and a substrate for a touch panel is particularly preferred. The material of the substrate for a touch panel is preferably glass or a transparent resin.

In addition, as the substrate, glass or a resin film used for an exterior part (excluding a display unit) in a device such as a mobile phone (for example, a smartphone), a portable information terminal (for example, a tablet terminal), a game machine, or a remote controller is also preferable.

The surface treatment agents containing the fluorine-containing compound are suitably used for applications in which it is required to maintain performance (friction resistance) in which water/oil repellency is less likely to decrease even when the surface layer is repeatedly rubbed with a finger and performance (fingerprint dirt removability) in which a fingerprint attached to the surface layer can be easily removed by wiping, for a long period of time, for example, as a surface treatment agent for a member constituting a surface touched by a finger of a touch panel, a spectacle lens, and a display of a wearable terminal.

### EXAMPLES

Hereinafter, the present invention will be described in detail using examples, but the present invention is not limited to these examples. In addition, Examples 1-1, 1-2, 2-3, and 2-4 are examples of the production method.

### Example 1

### Example 1-1

In a 250 mL recovery flask, 50 g of the compound 1-1 manufactured by

Tokyo Chemical Industry (TCI) Co., Ltd., 17 g of the compound 1-2, 150 g of 1,3-bis(trifluoromethyl)benzene, and 2 g of 2,2′-azobis(2-methylbutyronitrile) were added, and the mixture was stirred at 80° C. for 16 hours. The obtained crude liquid was concentrated and then purified by silica gel column chromatography to obtain 50 g of the compound 1-3.

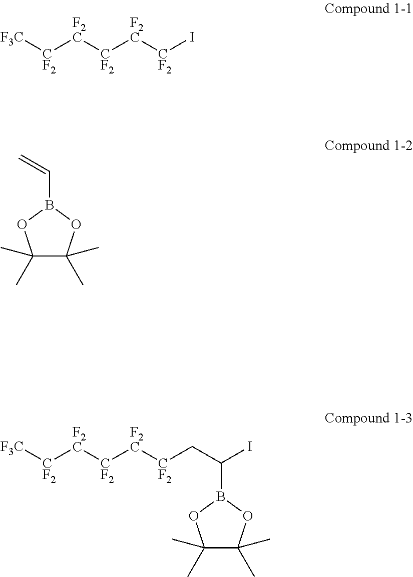

### Example 1-2

In a 50 mL recovery flask, 4 g of the compound 1-3 and 18 g of tetrahydrofuran were added, and the mixture was stirred at −78° C. to be dissolved. Then, 9 g of butenyl-magnesium bromide (1.0 mol/L tetrahydrofuran solution) was added, and the mixture was stirred for 18 hours while slowly raising the temperature to room temperature. Thereafter, hydrochloric acid was added, the mixture was extracted with methylene chloride, and the organic layer was concentrated and purified by silica gel column chromatography to obtain 2 g of the compound 1-4.

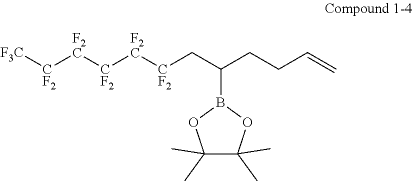 [SMILES: C=CCCC(CC(F)(F)C(F)(F)C(F)(F)C(F)(F)C(F)(F)C(F)(F)F)B1OC(C)(C)C(C)(C)O1]

### Example 1-3

In a 50 mL recovery flask, 2 g of the compound 1-4, 10 g of 1,3-bis(trifluoromethyl)benzene, 6 g of tetrahydrofuran, 0.5 g of acetic acid, and 0.5 g of triethylamine were added, and the mixture was stirred at 30° C. for 16 hours. The obtained crude liquid was concentrated and then purified by column chromatography to obtain 1.1 g of the compound 1-5.

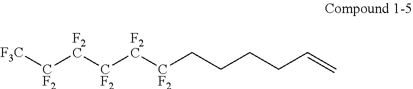 [SMILES: C=CCCCCC(F)(F)C(F)(F)C(F)(F)C(F)(F)C(F)(F)C(F)(F)F]

### Example 2

### Example 2-1

The following compound 2-1 was obtained according to the method described in Examples 11-1 to 11-3 of International Patent Publication No. WO 2013/121984.

CF₃—O—(CF₂CF₂O—CF₂CF₂CF₂CF₂O)ₙCF₂CF₂O—CF₂CF₂CF₂—CF₂OC(O)CF(CF₃)OCF₂CF₂CF₃ Formula 2-1

Average value of the number of units n: 13

### Example 2-2

In a 500 mL recovery flask shielded from light with aluminum foil, 5.8 g of sodium pyrithione and 100 mL of 1,3-bistrifluoromethylbenzene(trade name: SR-solvent) were input, and the mixture was stirred under ice cooling. Then, g of the compound 2-1 was slowly input, and the mixture was stirred for 2 hours while being ice-cooled. Next, 12.0 g of iodine and 1.8 g of 2,2-azobis(2-methylbutyronitrile) (trade name: V-59) were input, the aluminum foil shielded from light was removed, and the mixture was stirred at 85° C. overnight. The temperature was returned to 25° C., methanol was input, and the mixture was sufficiently stirred. Thereafter, AC-6000 was added to separate two layers, and the lower layer was collected and concentrated. The obtained crude product was purified by silica gel column chromatography to obtain 39.8 g of the following compound 2-2.

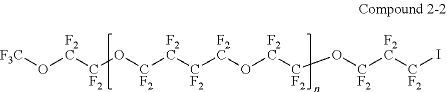

Average value of the number of units n: 13

### Example 2-3

In a 200 mL recovery flask, 1 g of the compound 2-2 10 g of the compound 1-2, 69 g of 1,3-bis(trifluoromethyl)benzene, and 0.5 g of 2,2′-azobis(2-methylbutyronitrile) were added, and the mixture was stirred at 80° C. for 18 hours. The obtained crude liquid was concentrated, then washed with methanol, and purified by silica gel column chromatography to obtain 10 g of the compound 2-3.

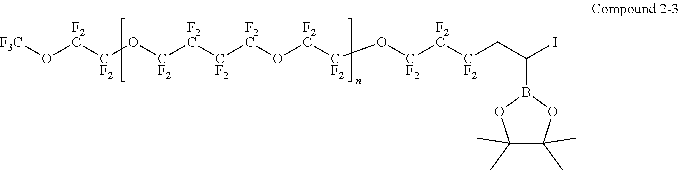

### Example 2-4

In a 50 mL recovery flask, 1 g of the compound 2-3, 10 g of 1,3-bis(trifluoromethyl)benzene, and 6 g of tetrahydrofuran were added, and the mixture was stirred at −78° C. to be dissolved. Then, 2.2 g of butenyl-magnesium bromide (1.0 mol/L THF solution) was added, and the mixture was stirred for 18 hours while slowly raising the temperature to room temperature. Thereafter, hydrochloric acid was added, the mixture was extracted with 1,3-bis(trifluoromethyl)benzene, and the organic layer was concentrated and purified by silica gel column chromatography to obtain 0.7 g of the compound 2-4. R in the following formula is CH₂CH₂CH═CH₂.

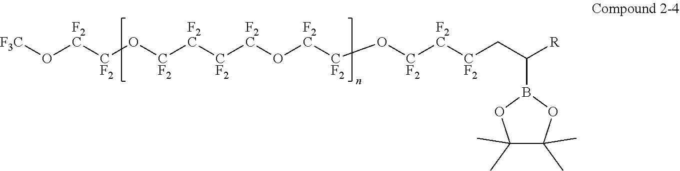

### Example 2-5

The following compound 2-5 was obtained in the same manner as in Example 1-3 except that the compound 1-4 was changed to the compound 2-4 in Example 1-3. R in the following formula is CH₂CH₂CH═CH₂.

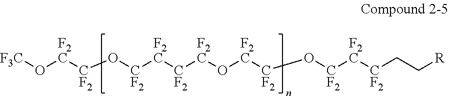

According to the present production method, any substituent can be introduced into a compound having a fluoroalkylene chain or a (poly)oxyfluoroalkylene chain under relatively mild reaction conditions using an easily available compound. The compound obtained by the present production method can be suitably used as, for example, a surface treatment agent capable of forming a surface layer having water/oil repellency, fingerprint wipe-off removability, and the like on a surface of a substrate, or a raw material thereof.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

1. A compound represented by the following formula (A1) or (A2):
G¹-(CH₂)ₘ₁—CHX¹—(CH₂)ₙ₁-M Formula (A1)
M-(CH₂)ₙ₂—CHX¹—(CH₂)ₘ₂-G²-(CH₂)ₘ₃—CHX¹—(CH₂)ₙ₃-M Formula (A2)
wherein in the formula (A1) and (A2),
G¹ is a fluoroalkyl group or a monovalent group having a (poly)oxyfluoroalkylene chain,
G² is a fluoroalkylene group or a divalent group having a (poly)oxyfluoroalkylene chain,
X¹ is a halogen atom, and when there are a plurality of X¹'s, the X¹'s may be the same as or different from each other,
M is BR¹R², R¹ and R² are each independently a group represented by —OH, —R¹¹, —OR¹¹, —O—C(O)—R¹¹ or —NR¹¹R¹²R¹¹ is a hydrocarbon group which may have a substituent or a heteroatom in a carbon chain, two R¹¹'s may be linked to form a ring structure, R¹² is a hydrogen atom or an alkyl group having 1 to 6 carbon atoms, and when there are a plurality of M's, the M's may be the same as or different from each other,
n1, n2, and n3 are each independently an integer of 0 to 20, and
m1, m2, and m3 are each independently 1 or 2.

2. A method for producing a compound represented by the following formula (B1) or (B2), wherein M in the compound according to claim 1 is converted into a hydrogen or hydrocarbon group represented by R³:
G¹-(CH₂)ₘ₁—CHX¹—(CH₂)ₙ₁—R³ Formula (B1)
R³—(CH₂)ₙ₂—CHX¹—(CH₂)ₘ₂-G²-(CH₂)ₘ₃—CHX¹—(CH₂)ₙ₃—R³ Formula (B2)
wherein in the formula (B1) or (B2),
G¹, G², X¹n1, n2, n3, m1, m2, and m3 are the same as in the formula (A1) or (A2), and
R³ is a hydrogen atom or a hydrocarbon group which may have a substituent or a heteroatom, and when there are a plurality of R³'s, the R³'s may be the same as or different from each other.

3. The method for producing a compound represented by the formula (B1) or (B2) according to claim 2, comprising:
reacting the compound represented by formula (A1) or (A2) with the compound represented by the following formula (C1):
R³—X² Formula (C1)
wherein in the formula (C1),
R³ is a hydrocarbon group which may have a substituent or a heteroatom, and
X² is a halogen atom.

4. A method for producing a compound represented by the following formula (B3) or (B4), wherein X¹ of the compound according to claim 1 is converted into a hydrogen or hydrocarbon group represented by R³:
G¹-(CH₂)ₘ₁—CHR³—(CH₂)ₙ₁-M Formula (B3)
M-(CH₂)ₙ₂—CHR³—(CH₂)ₘ₂-G²-(CH₂)ₘ₃—CHR³—(CH₂)ₙ₃-M Formula (B4)
wherein in the formula (B3) or (B4),
G¹, G², M, n1, n2, n3, m1, m2, and m3 are the same as in the formula (A1) or (A2), and
R³ is a hydrogen atom or a hydrocarbon group which may have a substituent or a heteroatom, and when there are a plurality of R³'s, the R³'s may be the same as or different from each other.

5. The method for producing a compound represented by the formula (B3) or (B4), according to claim 4, comprising:
reacting the compound represented by the formula (A1) or (A2) with a compound represented by the following formula (C2):
R³—MgR⁴ Formula (C2)
wherein in the formula (C2),
R³ is a hydrocarbon group which may have a substituent or a heteroatom, and
R⁴ is a halogen atom or a hydrocarbon group which may have a sub stituent or a heteroatom.

6. A method for producing the compound according to claim 1, comprising:
reacting a compound represented by the following formula (E1) or (E2) with a compound represented by the following formula (H1):
G¹-(CH₂)ₘ₄—X¹ Formula (E1)
X¹—(CH₂)ₘ₅-G²-(CH₂)ₘ₆—X¹ Formula (E2)
CH₂═CH—(CH₂)ₙ-M Formula (H1)
wherein in the formula (E1), (E2), and (H1),
G¹, G², X¹, and M, are the same as in the formula (A1) or (A2),
n is an integer of 0 to 20, and
m4, m5, and m6 are each independently 0 or 1.

7. A method for producing a surface treatment agent, comprising:
obtaining the compound represented by the formula (B1), or (B2) by the method according to claim 2; and
introducing a reactive silyl group into the compound.

8. A method for producing a surface treatment agent, comprising:
obtaining the compound represented by the formula (B1), or (B2) by the method according to claim 3; and
introducing a reactive silyl group into the compound.

9. A method for producing a surface treatment agent, comprising:
obtaining the compound represented by the formula (B3), or (B4) by the method according to claim 4; and
introducing a reactive silyl group into the compound.

10. A method for producing a surface treatment agent, comprising:
obtaining the compound represented by the formula (B3), or (B4) by the method according to claim 5; and
introducing a reactive silyl group into the compound.
